(19) United States
(12) Patent Application Publication
(10) Pub. No.: US 2008/0114401 A1
(43) Pub. Date: May 15, 2008

(54) Posterior Fixation Devices and Methods of Use

(75) Inventors: Mingyan Liu, Bourg la Reine (FR); Loic Josse, Denens (CH); Brice Edouard, Paris (FR); Jean-Charles LeHuec, Pessac (FR)

Correspondence Address: COATS & BENNETT/MEDTRONICS, 1400 CRESCENT GREEN, SUITE 300, CARY, NC, 27518, US

(73) Assignee: Warsaw Orthopedic, Inc., Warsaw, IN (US)

(21) Appl. No.: 11/558,511

(22) Filed: Nov. 10, 2006

(51) Int. Cl. A61B 17/70; A61B 17/88

(52) U.S. Cl. 606/276; 606/279

(57) ABSTRACT

The present application is directed to devices and methods for attaching one or more elongated members to a posterior surface of a vertebral member. The devices may include a body with a first face that contacts the vertebral member and a second opposite face. A hook may extend outward from the first face to engage the vertebral member. The body may also include one or more receivers to receive the elongated members. An engagement member may be movably connected to the body. The engagement member may include a first section that contacts the body, and a second section that includes a second hook. The engagement member may be positioned relative to the body to adjust a distance between the first and second hooks to attach the body to the vertebral member. A fastener may secure the engagement member to the body at the desired position. The fastener may also attach the one or more elongated members to the body.

### BACKGROUND

The present application is directed to posterior fixation devices and, more particularly, to devices that attach to a posterior surface of a vertebral members for securing one or more elongated members.

The spine is divided into four regions comprising the cervical, thoracic, lumbar, and sacrococcygeal regions. The cervical region includes the top seven vertebral members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The vertebral members of the spine are aligned in a curved configuration that includes a cervical curve, thoracic curve, and lumbosacral curve.

Various conditions may lead to damage of the vertebral members and/or intervertebral discs. The damage may result from a variety of causes including a specific event such as trauma, a degenerative condition, a tumor, or infection. Damage to the intervertebral discs and vertebral members can lead to pain, neurological deficit, and/or loss of motion. Elongated members, such as but not limited to rods, bars, and blades, may extend along the spine to redistribute stresses and/or restore proper alignment of the vertebral members. The elongated members may be substantially straight, or include a curved configuration to conform to the curvature of the spine.

The elongated members should be securely fixed to one or more of the vertebral members. Fixation often proves difficult because of the varied shape and dimensions of the vertebral members.

### SUMMARY

The present application is directed to devices and methods for attaching one or more elongated members to a posterior surface of a vertebral member. The devices may include a body with a first face that contacts the vertebral member and a second opposite face. A hook may extend outward from the first face to engage the vertebral member. The body may also include one or more receivers to receive the elongated members. An engagement member may be movably connected to the body. The engagement member may include a first section that contacts the body, and a second section that includes a second hook. The engagement member may be positioned relative to the body to adjust a distance between the first and second hooks to attach the body to the vertebral member. A fastener may secure the engagement member to the body at the desired position. The fastener may also attach the one or more elongated members to the body.

### BRIEF DESCRIPTION OF THE DRAWINGS

FIG. 1 is a side view according to one embodiment of a fixation system featuring a plurality of bodies that attach a pair of elongated members to vertebral members.

FIG. 2 is a perspective view from a first direction of a fixation device according to one embodiment.

FIG. 3 is a perspective view from a second direction of a fixation device according to one embodiment.

FIG. 4 is a perspective view from a third direction of a fixation device according to one embodiment.

FIG. 5 is a perspective view of a fixation device according to one embodiment.

FIG. 6 is a perspective view of a fixation device according to one embodiment.

FIG. 7 is an exploded perspective view of a fixation device according to one embodiment.

FIG. 8 is a side view of a fixation system in one context of use according to one embodiment.

FIG. 9 is a side view of a fixation system in one context of use according to one embodiment.

FIG. 10 is a side view of a fixation system in one context of use according to one embodiment.

### DETAILED DESCRIPTION

The present application is directed to fixation devices and systems for attaching one or more elongated members to one or more vertebral members. The fixation devices include a body with hooks that cooperate to attach to the vertebral member. The body also includes one or more receivers for receiving the elongated members. A fastener may attach to the body to secure the position of the hooks, and the one or more elongated members to the body. The devices may be used on two or more vertebral members to form a fixation system. The fixation system extends along a section of the spine to position and/or redistribute stresses to the vertebral members.

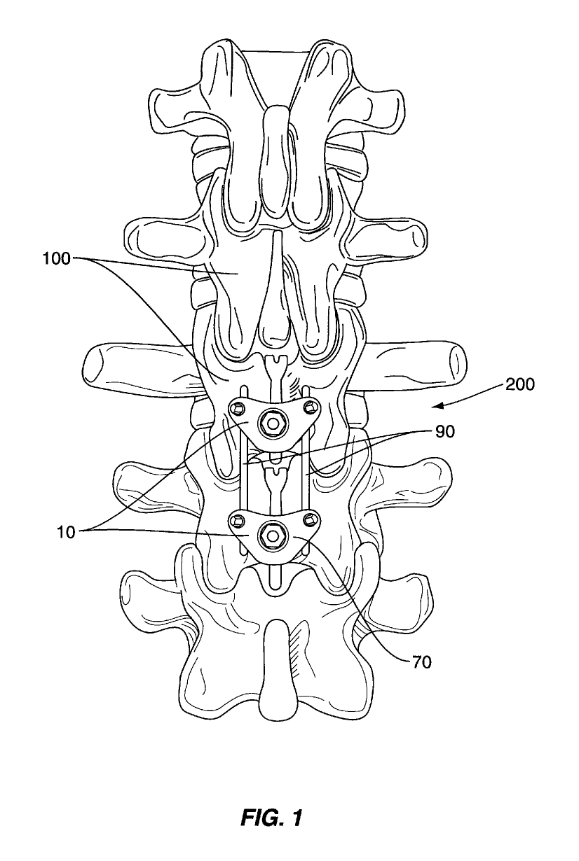

FIG. 1 illustrates one embodiment of a pair of fixation devices 10 that each attach to a posterior section of the vertebral members 100. The devices 10 may be positioned on the posterior surface of the vertebral members 100 and include hooks that attach to the lamina. The devices 10 further include one or more receivers to receive elongated members 90. Two or more devices 10 may be attached to the vertebral members 100 to anchor the elongated members 90 and form a fixation system 200. The fixation system 200 supports and/or positions the vertebral members 100.

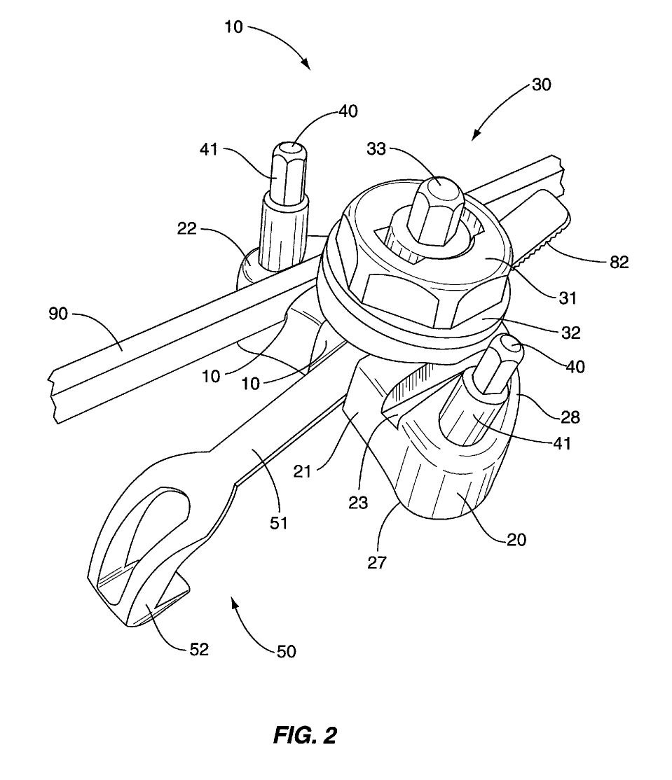

Each of the fixation devices 10 includes a body 20 that extends across a section of the vertebral member 100. As illustrated in FIGS. 2, 3, and 4, body 20 includes a first section 21 and a pair of lateral wings 22. The lateral length of the body 20 measured between the outer edges of the wings 22 may vary depending upon the context of use. Body 20 may further include a curved shape with the inner surface 27 of the lateral wings 22 being offset from the first section 21. The curved shape of the body 20 conforms to the posterior surface of the vertebral member 100 with the first section being positioned substantially at a centerline of the vertebral member 100, and the lateral wings 22 positioned on each side of the centerline.

The body 20 includes the inner surface 27 that contacts the posterior surface of the vertebral member 100 (i.e., faces in an anterior direction) and an outer surface 28 that faces away from the vertebral member 100 (i.e., faces in a posterior direction). One or more slots 23 may extend into the outer surface 28 to receive the elongated members 90. The slots 23 may be positioned at a variety of distances outward from the first section 21. The slots 23 may further include a variety of shapes and widths to receive the elongated members 90. Slots 23 may include a depth such that the elongated member 90 is recessed below the outer surface 28 when positioned within the slot 23, or include a depth so the elongated member 90 may extend above the outer surface 28.

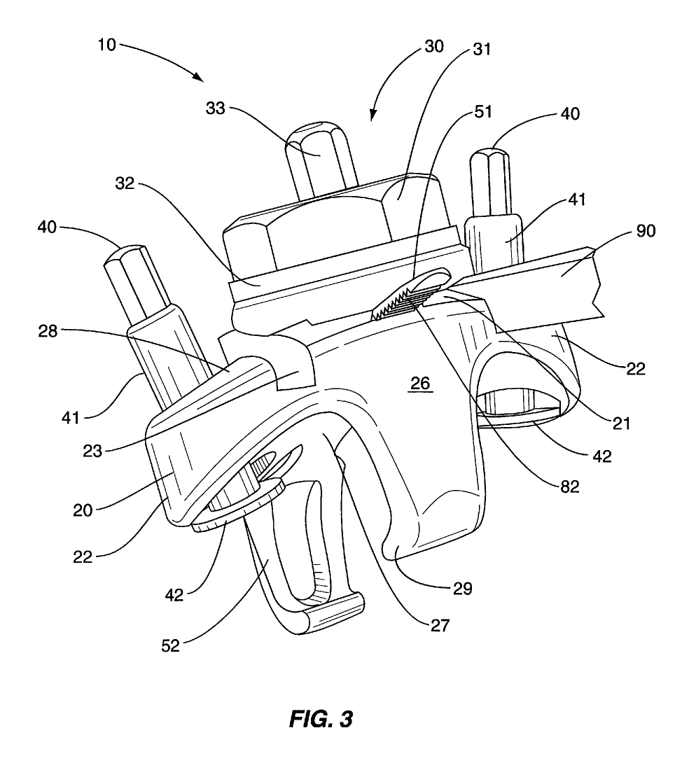

In one embodiment as illustrated in FIGS. 2 and 3, a lateral slot 23 is positioned on each of the wings 22. In another embodiment, a single slot 23 is positioned on one of the wings 22. In yet another embodiment, three or more slots 23 are positioned on the wings 22. In one multiple slot embodiment, the slots 23 are substantially parallel.

Body 20 further includes a hook 25 that extends outward from the inner surface 27. Hook 25 may extend outward from an edge of the body 20. In one embodiment, hook 25 extends outward from the superior edge of the body 20 to contact against an edge of the vertebral member 100. Hook 25 may also be laterally aligned with the first section 21 of the body 20 and may be substantially aligned with a centerline of the vertebral member 100 when the body 20 is attached.

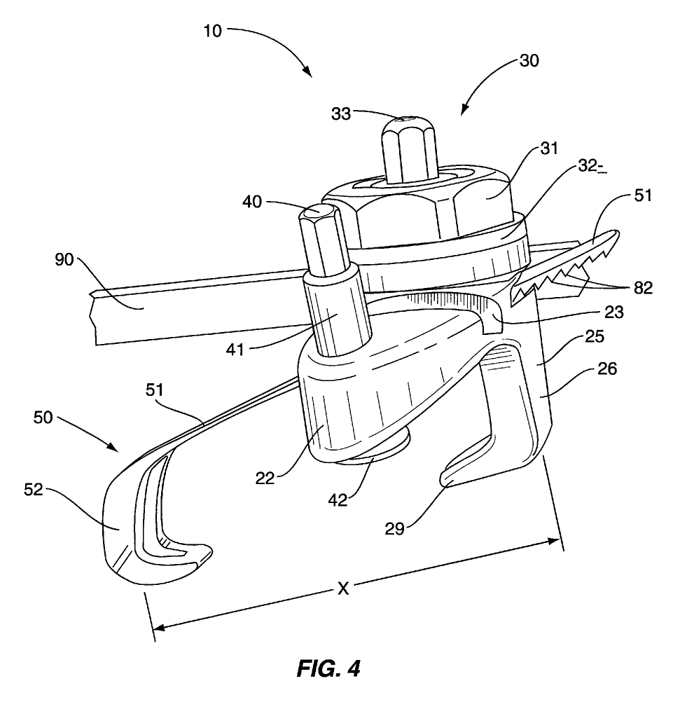

The hook 25 includes a first section 26 that extends outward from the inner surface 27, and a second section 29 that extends at an angle from the end of the first section 26. The hook 25 is constructed for the first section 26 to extend along a superior edge of the lamina of the vertebral member 100, and the second section 29 to extend along an anterior edge of the lamina. As illustrated in FIG. 4, the second section 29 may include a ramped thickness that decreases outward from the first section 26. The ramped shape facilitates positioning the second section 29 along the lamina and attaching the hook 25 to the vertebral member 100.

The body 20 may further include a slot 24 that extends into the outer surface 28 of the first section 21. Slot 24 may be angled with a ramped lower surface that extends within the body 20 between a first sidewall and the outer surface 28. In one embodiment, slot 24 extends through the sidewall and terminates within a central portion of the body 20. In another embodiment, slot 24 extends within the body 20 between the first sidewall and a second, opposite sidewall. The slot 24 is further aligned with the hook 25 and is sized to receive an engagement member 50.

The engagement member 50 is movably attached to body 20. One embodiment of the engagement member 50 is illustrated in FIGS. 2-4 and includes an arm 51 and a hook 52. Arm 51 contacts the body 20 and is sized to fit and move within the slot 24. The arm 51 may include a width that is substantially the same as the slot 24 to prevent lateral movement of the hook 50. The arm 51 provides for adjusting a distance X between the hook 25, 52 as illustrated in FIG. 4. Arm 51 may include a length that is greater than the body 20 resulting in an end of the arm 51 that is opposite from the hook 52 to extend outward beyond the body 20. Teeth 82 may be positioned on the underside of the arm 51 to mate with corresponding teeth within the slot 24. The teeth 82 maintain the position of the arm 51 within the slot and prevent sliding that may inadvertently change the distance X.

Hook 52 is shaped to engage a side of the vertebral member 100. Hook 52 works in combination with hook 25 to press in opposite directions and hold the central portion of the laminar. This forms a clamp that is stable in cephalad-caudal and pull-out directions. Hook 52 may include a variety of shapes and sizes similar to the hook 25 described above. Hook 52 may be axially aligned with the arm 51 as illustrated in FIGS. 2-4. In one embodiment, the hook 52 and arm 51 are substantially aligned with the centerline of the vertebral member 100.

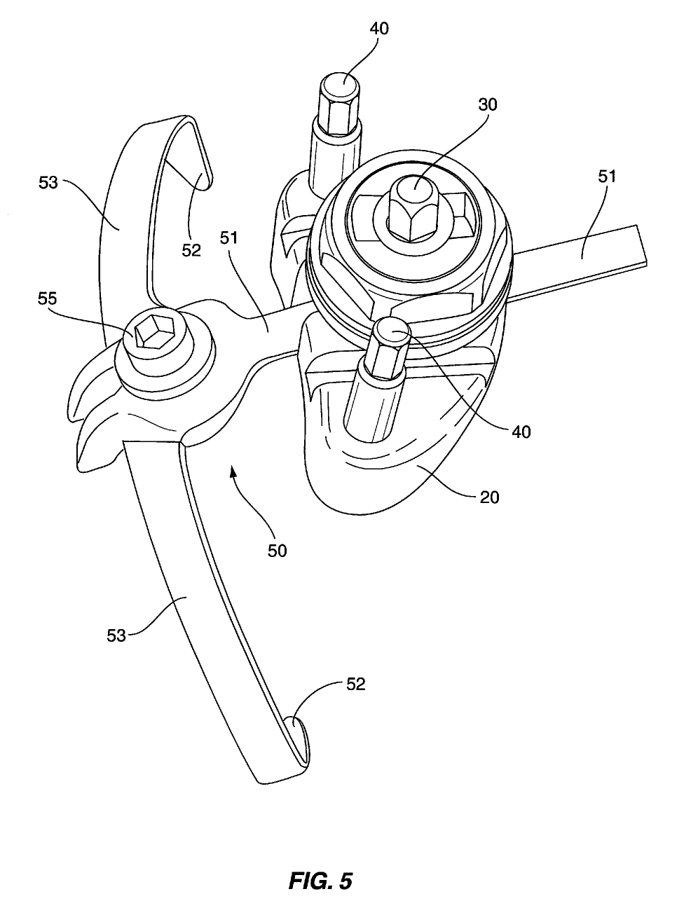

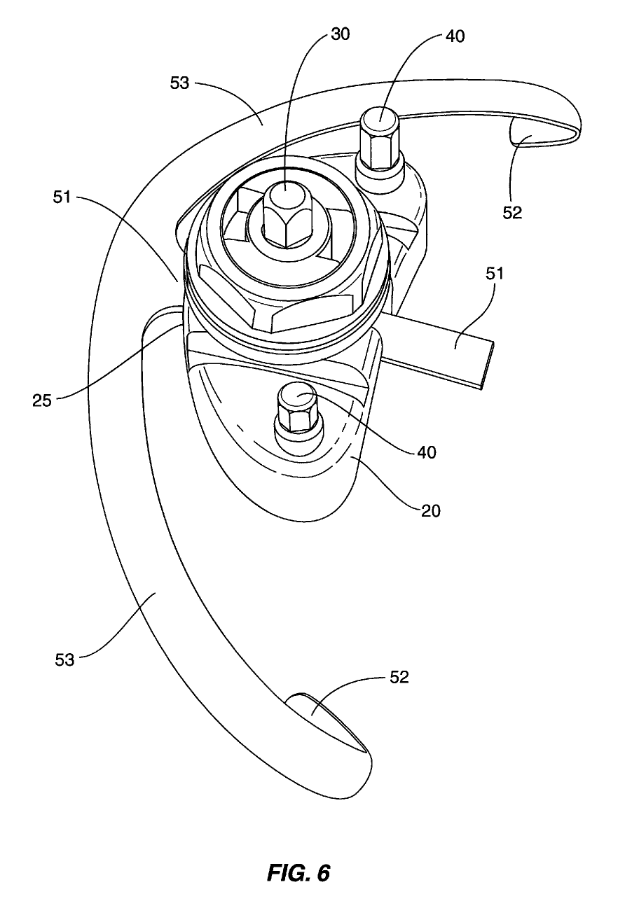

The engagement member 50 may also include other shapes and sizes to attach to the vertebral members 100. FIG. 5 illustrates an engagement member 50 that includes a first arm 51 that attaches to the body 20, and a pair of second arms 53. Second arms 53 extend outward from the arm 51 and include hooks 52 for engaging with the vertebral member 100. In one embodiment as illustrated, second arms 53 extend outward from arm 51 at substantially 90° to from a substantially T-shape. This embodiment may be effective for engaging a vertebral member 100 in the lumbar region with the hooks 52 positioned to engage the pars interarticularis. Arms 53 may also extend outward at other angles as necessary. Further, the arms 53 may extend outward at the same or different angles. FIG. 6 illustrates another embodiment with arms 53 including a curved configuration each with a hook 52 at the end.

The engagement member 50 may be constructed as a single piece, or multiple pieces. In the embodiments of FIGS. 5 and 6, multiple-piece designs may be constructed of two pieces with arm 51 and one of arms 53 formed as a first piece, or as three pieces with each arm 53 being separate from arm 51. In multiple-piece designs, a fastener 55 may connect together the separate pieces. Fastener 55 may include a variety of configurations including but not limited to a nut and bolt combination, a screw, a pin, and a rivet. Fastener 55 may extend through apertures in the arms 51, 53. The apertures may be sized to adjust the angular position of each arm 53. Further, the apertures may allow for adjusting the distance between the hooks 52.

Body 20 may be attached to the vertebral member 100 in a variety of configurations. In one manner, body 20 is positioned for the hook 25 to engage a superior edge of the vertebral member 100. Another manner includes the body 20 being positioned with the hook 25 engaging an inferior edge.

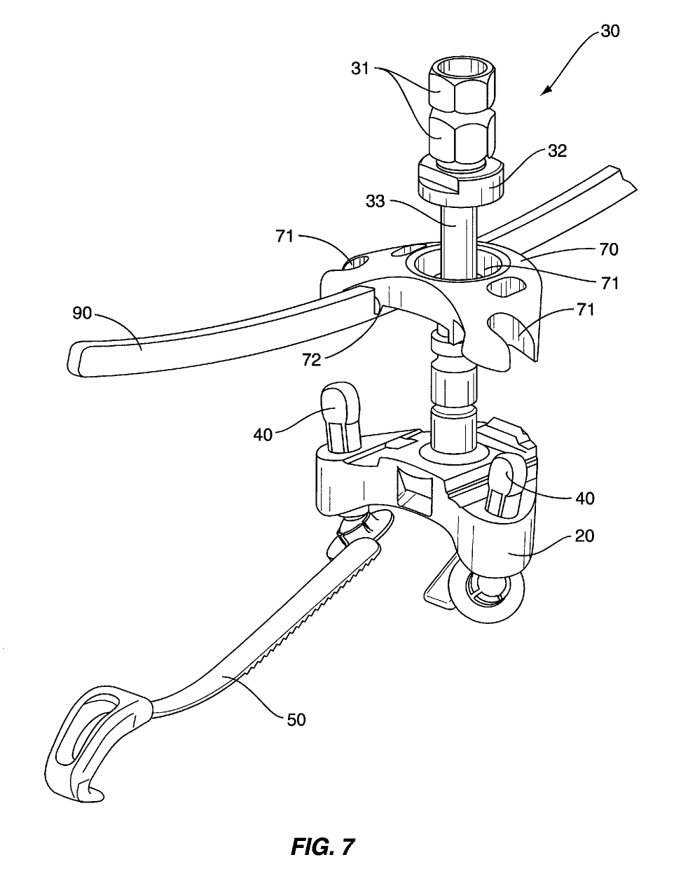

The engagement member 50 is secured to the body 20 with a fastener 30. In one embodiment, fastener 30 includes one or more fixation members 31 that attach at the outer surface 28 of the body 20. In one embodiment as illustrated in FIGS. 2 and 7, fixation member 31 is a nut mounted on a threaded bolt 33 that extends outward from the body 20. Rotation of the nut adjusts the amount of compressive force applied to the engagement member 50. In another embodiment, fixation member 31 includes an integral head and a threaded shaft. The threaded shaft mates within a threaded aperture in the body 20. Rotation of the member 31 causes the shaft to engage within the aperture and the head to apply a compressive force to the engagement member 50. The fixation member 31 may include a polygonal outer shape to receive a tool for rotating the fixation member 31.

A capture member 32 may also be position below the fixation member 31. In one embodiment, capture member 32 includes a central opening sized to fit around the threaded bolt 33. Capture member 32 includes a width to extend across the entirety or a limited section of the slots 23 to maintain the elongated members 90 within the slots 23. Capture member 32 may include a width that is substantially the same or greater than the fixation member 31. In one embodiment, the capture member 32 extends to the slots 32, but the fixation member 31 includes a shortened width that does not extend to the slots 23. Therefore, the capture member 32 maintains the members 90 within slots 23, but the fixation nut 31 does not.

The threaded bolt 33 may include an extended length as illustrated in FIG. 7. The length allows the bolt 33 to act as a guide to connect the elongated members 90 with the body 20. After attachment, the bolt 33 may be cut to a smaller length.

One or more attachment members 40 may further attach the body 20 to the vertebral member 100. Attachment members 40 include a shaft 41 that extends from the body 20, and a pivoting foot 42 at the lower end to contact the posterior surface of the vertebral member 100. The shaft 41 fits within an aperture that extends through the body 20. Shaft 41 may include threads with rotation causing the shaft to move into and out of the body 20. An outer end of the shaft 41 may further include a tool engaging surface, such as a polygonal configuration. Feet 42 are pivotally attached to an inner end of the shaft 41 and are adjustable to accommodate the varied surface of the vertebral member 100. Feet 42 may include an enlarged contact area for increased contact with the posterior surface of the vertebral member 100. In one embodiment, attachment members 40 are positioned at each of the wings 22. In one embodiment, an attachment member 40 is positioned on each of the wings 22. The members 40 work in combination to control the positioning of the body 20.

The stability of the device 10 is based on a multi-directional equilibrium of control forces from the various attachment points. In one embodiment, hooks 25, 52 press in opposite directions and hold the central portion of the laminar, while opposing attachment members 40 press down to the posterior surface of the laminar. The combination of the hooks 25, 52 and attachment members 40 actively controls both anterior-posterior and medial-lateral movements. The relative positioning of these members also controls any rotational movement.

Installation of the device 10 on the posterior of the vertebral member 100 may initially require removal of the spinous process. The removal may be performed by various measures known in the art. Once removed, the body 20 is placed on the posterior surface of the vertebral member 100. Initially, the hook 25 may capture the vertebral member 100 with the first section 26 extending along a first edge of the vertebral member 10 and the second section 29 positioned a second edge of the vertebral member 100. With the hook 25 positioned, the remainder of the body 20 is aligned relative to the vertebral member 100. The first section 21 may be aligned on the centerline of the vertebral member 100, with the wings 22 extending laterally outward from the centerline. Further, the inner surface 27 is positioned in contact with the posterior surface of the vertebral member 100.

After positioning the body 20, the engagement member 50 is adjusted to cooperate with the hook 25. The one or more hooks 52 engage the vertebral member 100, and then the arm 51 is slid within the slot 24. The cooperation between the one or more hooks 52 and hook 25 captures the vertebral member. In one embodiment, the arm 51 may be moved an amount causing the hooks 25, 52 to apply a compressive force to the vertebral member 100. The one or more attachment members 40 may further be positioned with the feet 42 contacting the posterior surface to further solidify the attachment of the body 20.

The one or more elongated members 90 are placed with the slots 23 of the body 20. The fastener 30 may then be used to extend over the slots 23 and prevent removal of the elongated members 90. The fastener 30 further locks the position of the engagement member 50.

A cap 70 may attach to the outer surface 28 of the body 20. FIG. 7 illustrates an exploded view of the cap 70 positioned above the body 20, and FIG. 1 illustrates the cap 70 attached to the body 20. Cap 70 may be sized and shaped to substantially match the body 20. Cap 70 may include openings 71 sized to extend around the attachment members 40 and threaded bolt 33. In one embodiment, the fastener 30 attaches the cap 70 to the body 20.

Cap 70 may also include a connector 72 that attaches to the elongated member 90. Connector 72 may include a clip on an underside that extends around and attaches to the elongated member 90. In one method of attaching the elongated member 90 to the body 20, the member 90 is initially attached to the cap 70 and then fixed to the body 20 together with the cap 70. As illustrated in FIG. 7, the cap 70 may interact with the elongated bolt 33 to guide the elongated member 90 to the body 20.

The fixation system 200 may be comprised of one or more devices 10. The devices 10 may be attached to vertebral members 100 that are adjacent, or spaced apart. Further, the fixation system may include one or more elongated members 90 that extend along the spine.

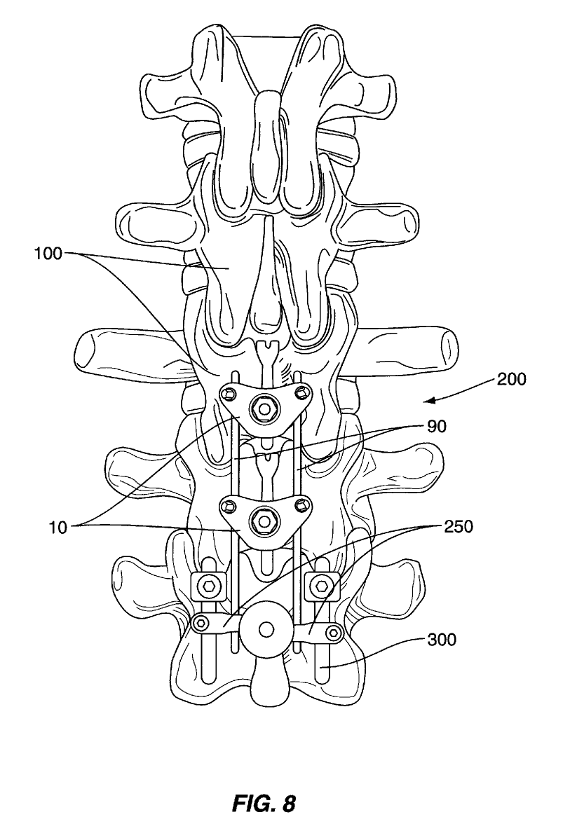

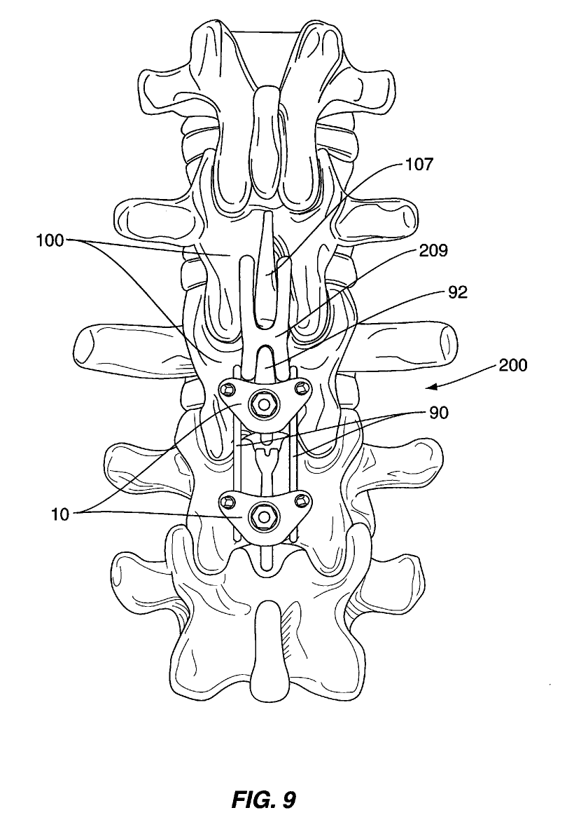

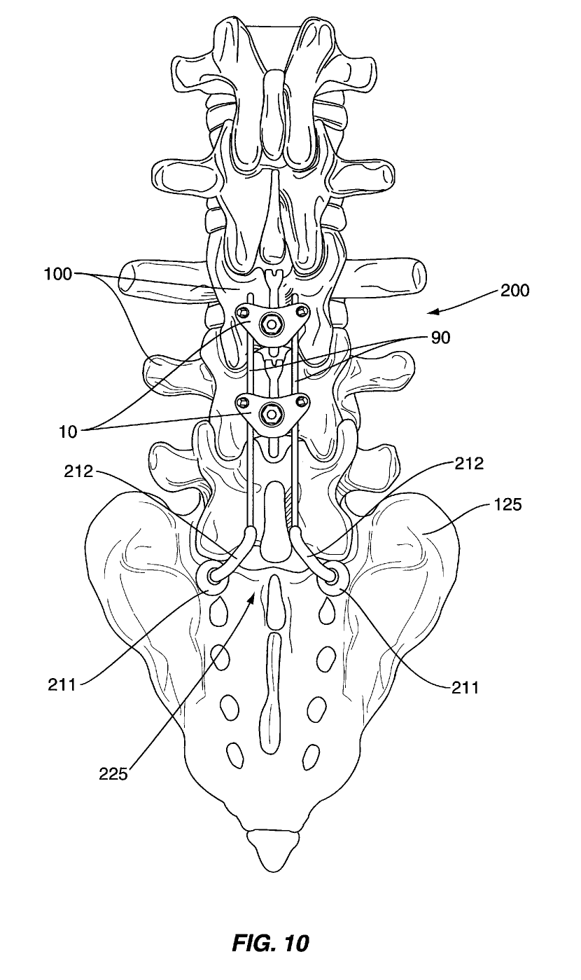

Fixation system 200 may be used alone, or in combination with other structures. FIG. 1 illustrates an embodiment with the system 200 being used by itself without any other structures. FIG. 8 illustrates the system 200 connected with a pedicle screw based system 300. In this embodiment, one or more connectors 250 connect the elongated members 90 with the additional system 300. FIG. 9 illustrates an embodiment used in combination with an interspinous spacer 209. A base 92 extends in a posterior direction from the device to support a first end of the spacer 209. The base 92 may be attached to one or both of the body 20 and the engagement member 50. The second end of the spacer 209 is positioned against a spinous process 107 of the vertebral member 100. FIG. 10 illustrates the fixation system 200 in combination with a sacral channel system 225. Legs 212 connect to the inferior ends of the elongated members 90. Plugs 211 at the ends of the legs 212 attach to the sacrum 125. Plugs 211 may include an exterior thread for insertion into the sacrum 125.

In some of the embodiments described above, the arm 51 of the engagement member 50 is positioned within a slot 24 in the body 20. In another embodiment, the arm 51 is simply placed on the outer surface 28 of the body 20. The fastener 30 applies a compressive force to capture the arm 51 against the outer surface 28. In one embodiment, the arm 51 includes a slot sized to receive the threaded bolt 33 or shaft of the fastener 30.

In one embodiment, the body 20 includes a first section 21 and a pair of wings 22 that extend laterally outward. In another embodiment, body 20 may include a single wing 22 extending outward from the first section 21. This embodiment may be used when the fixation system 200 anchors a single elongated member 90 to the vertebral members 100.

Spatially relative terms such as “under”, “below”, “lower”, “over”, “upper”, “superior”, “inferior”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A fixation device to attach an elongated member to a posterior surface of a vertebral member, the fixation device comprising:
a body including a first face that contacts the posterior surface of the vertebral member and a second face opposite the first face, the body further including a first hook that extends outward beyond the first face to engage the vertebral member;
a receiver formed within the body to receive the elongated member;
an engagement member including a second hook, the engagement member being slidably attached to the body to adjust a distance between the first hook and the second hook to apply a compressive force to engage the vertebral member.

2. The device of claim 1, wherein the first hook is aligned along a center of the body.

3. The device of claim 2, wherein the second hook is axially aligned with the first hook and faces towards the first hook.

4. The device of claim 1, wherein the engagement member extends outward from the body in a common direction as the first hook.

5. The device of claim 1, wherein the engagement member extends outward from the body in a direction opposite from the first hook.

6. The device of claim 1, wherein the engagement member is substantially T-shaped with a central member and a pair of arms that each includes a hooked end.

7. The device of claim 1, further comprising attachment members that extend through the body and include feet that contact the posterior surface of the vertebral member.

8. The device of claim 1, further comprising a slot that extends into the second face of the body and is sized to receive an arm of the engagement member.

9. The device of claim 1, further comprising a fastener that attaches to the body and extends over the receiver to secure the elongated member to the body and extends over the engagement member to secure the engagement member to the body.

10. The device of claim 1, further comprising a cap that attaches to the second face of the body and extends over the elongated member.

11. A fixation device to attach an elongated member to a posterior surface of a vertebral member, the fixation device comprising:
a body including a first face that contacts the posterior surface of the vertebral member and a second face opposite the first face, the body further including a first hook that extends outward beyond the first face to engage the vertebral member;
a receiver formed within the body to receive the elongated member;
an engagement member including a first section and a second section, the first section being slidably attached to the second face of the body and being movable to adjust a distance between the first hook and the second section; and
a fastener to secure the first section to the body and prevent movement between the engagement member and the body.

12. The device of claim 11, wherein the fastener further secures the elongated member within the receiver.

13. The device of claim 11, wherein the second section includes a second hook that is axially aligned with the first hook when the engagement member is secured to the body.

14. The device of claim 11, further comprising a cap that attaches to the second face of the body and extends over the elongated member and contain the elongated member within the receiver.

15. The device of claim 11, wherein the second section is curved and includes hooked ends.

16. The device of claim 11, further comprising a second receiver to receive a second elongated member, the second receiver being substantially parallel with the receiver.

17. A fixation device to attach first and second elongated members to a posterior surface of a vertebral member, the fixation device comprising:
a body including a first face that contacts the posterior surface of the vertebral member and a second face opposite the first face, the body including a central section and first and second wings that extend laterally outward from opposing sides of the central section, the body further including a first hook that extends outward from the central section and beyond the first face to engage the vertebral member;
a first receiver formed within the first wing to receive the first elongated member;
a second receiver formed within the second wing to receive the second elongated member;
an engagement member that includes a second hook, the engagement member operatively attached to the central section and being movable to adjust a distance between the first hook and the second hook; and
a fastener attached to the body and sized to extend across the first and second receivers to secure the first and second elongated members and the engagement member to the body.

18. The device of claim 17, wherein the engagement member is substantially straight and the second hook is axially aligned with the first hook.

19. The device of claim 17, wherein the fastener comprises a nut and a threaded bolt.

20. The device of claim 17, further comprising a cap that extends over the elongated member and the body and is attached to the body by the fastener.

21. The device of claim 17, wherein the first hook and the engagement member each extend outward from a common side of the body.

22. The device of claim 17, wherein the engagement member includes a first section that contacts the body and a second section, the second section includes a pair of arms that each includes hooked ends.

23. A method of attaching an elongated member to a posterior surface of a vertebral member, the method comprising the steps of:
positioning a body on the vertebral member with a first face contacting the posterior surface and a second face facing outward;
attaching a first hook that extends outward beyond the first face to the vertebral member;
moving an engagement member that is operatively connected to the body and adjusting a distance between a second hook on the engagement member with the first hook;
positioning the elongated member within a receiver on the second face of the body; and
attaching a fastener to the second face and securing the engagement member to the body and the elongated member within the receiver.

24. The method of claim 23, wherein the step of moving the engagement member that is operatively connected to the body comprises sliding a first section of the engagement within a slot in the second face of the body.

25. The method of claim 23, further comprising adjusting a distance between the second hook and a third hook on the engagement member.

26. The method of claim 23, further comprising aligning the first hook and the second hook along a centerline of the vertebral member.

27. The method of claim 23, wherein the step of attaching the fastener to the second face comprises attaching a nut to a threaded bolt.

28. The method of claim 23, wherein the step of positioning the elongated member within the receiver on the second face of the body comprises attaching the elongated member to a cap and guiding to the cap and elongated member to the body.

29. A method of attaching first and second elongated members to a posterior surface of a vertebral member, the method comprising the steps of:
positioning a body on the vertebral member with a first face contacting the posterior surface and a second face facing outward;
attaching a first hook that extends outward beyond the first face to the vertebral member;
attaching the body to the vertebral member by moving an engagement member operatively connected to the body and adjusting a distance between a second hook on the engagement member with the first hook;
positioning the first elongated member within a first receiver on the second face of the body and positioning the second elongated member within a second receiver on the second face of the body; and
attaching a fastener to the second face of the body to extend over at least a portion of the engagement member and the first and second receivers.
